(19) United States
(12) Patent Application Publication
(10) Pub. No.: US 2013/0116562 A1
(43) Pub. Date: May 9, 2013

(54) METHOD AND APPARATUS FOR GENERATING DIAGNOSTIC IMAGE AND MEDICAL IMAGE SYSTEM

(71) Applicants: Dong-geon KONG, Yongin-si (KR); Ki-wan CHOI, Yongin-si (KR)

(72) Inventors: Dong-geon KONG, Yongin-si (KR); Ki-wan CHOI, Yongin-si (KR)

(73) Assignee: Samsung Electronics Co., Ltd., Suwon-si (KR)

(21) Appl. No.: 13/557,691

(22) Filed: Jul. 25, 2012

(30) Foreign Application Priority Data
Nov. 9, 2011 (KR) 10-2011-0116472

(51) Int. Cl. A61B 8/14; A61N 7/02

(52) U.S. Cl. 600/439; 600/443

(57) ABSTRACT

A method of generating a diagnostic image of a subject including transmitting a transmission signal to the subject; acquiring a first RF frame and a second RF frame from an echo signal reflected from the subject; estimating a second directional displacement indicating a degree of movement of a first point of the subject appearing in the first RF frame in the second RF frame; generating an ultrasonic image corresponding to the second RF frame; and correcting an error of the generated ultrasonic image using the estimated second directional displacement. The first point may appear in the first RF frame and the second RF frame.

### CROSS-REFERENCE TO RELATED APPLICATIONS

This application claims the priority benefit of Korean Patent Application No. 10-2011-0116472, filed on Nov. 9, 2011, in the Korean Intellectual Property Office, the disclosure of which is incorporated herein in its entirety by reference.

### BACKGROUND

1. Field

Embodiments relate to a method and apparatus for generating a diagnostic image and a medical image system.

2. Description of the Related Art

An ultrasonic image of a subject may be generated by transmitting an ultrasonic signal to the subject and using an echo signal reflected from the subject. In this regard, the ultrasonic image of the subject may include a temperature image indicating a temperature of a cross-section of the subject or a brightness (B)-mode image indicating brightness of the cross-section of the subject. Further, a propagation speed of the ultrasonic signal used to generate the ultrasonic image differs according to a temperature of a medium.

### SUMMARY

According to an aspect of one or more embodiments, there is provided methods and apparatuses for generating a diagnostic image and a medical image system having an improved accuracy.

According to an aspect of one or more embodiments, there is provided a non-transitory computer readable recording medium having recorded thereon a program for executing the method.

According to an aspect of one or more embodiments, there is provided a method of generating a diagnostic image with respect to a first direction and a second direction of a subject, the method including: transmitting a transmission signal in the first direction to the subject; acquiring at least two radio frequency (RF) frames including a first RF frame and a second RF frame from an echo signal reflected from the subject; estimating a second directional displacement indicating a degree of movement of a first point of the subject appearing in the first RF frame in a second direction in the second RF frame; generating an ultrasonic image corresponding to the second RF frame; and correcting an error of the generated ultrasonic image using the estimated second directional displacement.

According to an aspect of one or more embodiments, there is provided a non-transitory computer readable recording medium having recorded thereon a program for executing the method of generating a diagnostic image.

According to an aspect of one or more embodiments, there is provided an apparatus for generating a diagnostic image with respect to a first direction and a second direction of a subject, the apparatus including: at least one transducer to transmit a transmission signal in the first direction to the subject and receiving an echo signal reflected from the subject; an RF frame acquisition unit to acquire at least two RF frames including a first RF frame and a second RF frame from the echo signal; a displacement estimation unit to estimate a second directional displacement indicating a degree of movement of a first point of the subject appearing in the first RF frame in a second direction in the second RF frame; an image generation unit to generate an ultrasonic image corresponding to the second RF frame; and an error correction unit to correct an error of the generated ultrasonic image using the estimated second directional displacement.

According to an aspect of one or more embodiments, there is provided a medical image system including: an apparatus to generate a diagnostic image by transmitting a transmission signal in a first direction to a subject, to acquire a first radio frequency (RF) frame and a second RF frame from an echo signal reflected from the subject, to estimate a second directional displacement indicating a degree of movement of a first point of the subject appearing in the first RF frame in a second direction perpendicular to the first direction in the second RF frame, and to generate an error corrected ultrasonic image using the estimated second directional displacement; and a display unit to display the generated error corrected ultrasonic image.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and/or other aspects will become apparent and more readily appreciated from the following description of embodiments, taken in conjunction with the accompanying drawings of which:

FIG. 1 is a block diagram of an apparatus for generating a diagnostic image, according to an embodiment;

FIG. 2 illustrates a propagation path of an ultrasonic signal and a propagation speed thereof with respect to a temperature of a medium, according to an embodiment;

FIG. 3 is a diagram for explaining a method of estimating a displacement in a displacement estimation unit of FIG. 1;

FIG. 4A is a diagram for explaining a method of extracting a second directional analytic signal so as to calculate cross-correlation in a displacement estimation unit of FIG. 1;

FIG. 4B is a diagram for explaining a method of calculating cross-correlation between a first radio frequency (RF) frame and a second RF frame in a displacement estimation unit of FIG. 1;

FIG. 5 is a diagram for explaining a method of estimating a displacement in a displacement estimation unit of FIG. 1;

FIG. 6 is a diagram for explaining a method of correcting an error of an ultrasonic image in an error correction unit of FIG. 1;

FIG. 7 is a block diagram of a medical image system, according to an embodiment; and

FIG. 8 is a flowchart illustrating a method of generating a diagnostic image, according to an embodiment.

### DETAILED DESCRIPTION

Reference will now be made in detail to embodiments, examples of which are illustrated in the accompanying drawings, wherein like reference numerals refer to the like elements throughout. In this regard, embodiments may have different forms and should not be construed as being limited to the descriptions set forth herein. Accordingly, embodiments are merely described below, by referring to the figures, to explain aspects of the disclosure.

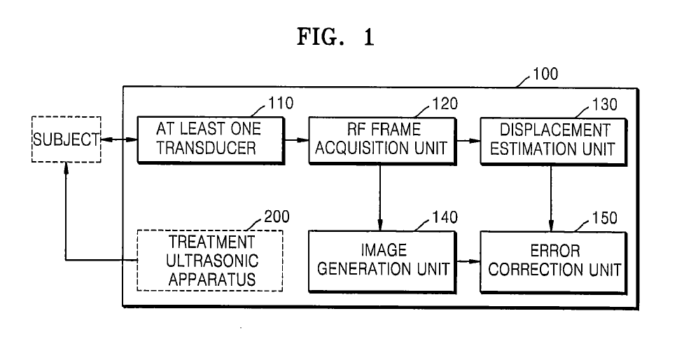

FIG. 1 is a block diagram of an apparatus 100 for generating a diagnostic image, according to an embodiment. Referring to FIG. 1, the apparatus 100 for generating the diagnostic image includes at least one transducer 110, a radio frequency (RF) frame acquisition unit (radio frequency frame acquirer) 120, a displacement estimation unit (displacement estimator) 130, an image generation unit (image generator) 140, and an error correction unit (error corrector) 150.

Elements related to an embodiment are shown in the apparatus 100 for generating the diagnostic image of FIG. 1. In addition, other general-purpose elements may be further included in the apparatus 100 for generating the diagnostic image.

Also, the RF frame acquisition unit 120, the displacement estimation unit 130, the image generation unit 140, and the error correction unit 150 of FIG. 1 may include one or more processors. A processor may be realized in an array of a plurality of logic gates, or in a combination of a general-purpose microprocessor and a memory storing a program executable in the general-purpose microprocessor. However, a processor may be realized in other types of hardware.

The apparatus 100 for generating the diagnostic image generates the diagnostic image of a subject in a first direction and a second direction. For example, the subject may include a predetermined treatment part to which heat is applied. The predetermined treatment part according to an embodiment may include a tumor.

More specifically, although the apparatus 100 for generating the diagnostic image may further include a treatment ultrasonic apparatus 200 for applying heat, the treatment ultrasonic apparatus 200 may be disposed outside the apparatus 100 for generating the diagnostic image according to a usage environment.

For example, the treatment ultrasonic apparatus 200 may apply heat to a treatment part of the subject as a treatment ultrasonic signal is irradiated onto the treatment part of the subject. Accordingly, the apparatus 100 for generating the diagnostic image according to an embodiment may be a diagnostic ultrasonic apparatus for transmitting and receiving a diagnostic ultrasonic signal in a high intensity focused ultrasound (HIFU) system but is not limited thereto.

Further, the subject according to an embodiment may include all organs of a human body such as a liver, an abdomen, a heart, a brain, etc. The diagnostic image may include a brightness (B)-mode image, a temperature image, etc. as an image of the subject generated using the ultrasonic signal but is not limited thereto.

The diagnostic image according to an embodiment may include information regarding the first direction and information regarding the second direction. For example, the diagnostic image may be a diagnostic image of cross-sections of the subject formed in the first direction and the second direction.

In this regard, the first direction may be an ultrasonic propagation direction of the subject. For example, the first direction may include an axial direction or a depth direction, etc. The second direction may be a direction perpendicular to the ultrasonic propagation direction. For example, the second direction may include a lateral direction.

The at least one transducer 110 transmits a transmission signal with respect to the subject in the first direction, and receives an echo signal reflected from the subject. The at least one transducer 110 may be a first dimensional transducer-array or a second dimensional transducer-array but is not limited thereto. The at least one transducer 110 according to an embodiment may be included in a probe but is not limited thereto.

The at least one transducer 110 converts an electrical signal into an ultrasonic signal, transmits the converted ultrasonic signal to the subject, receives the ultrasonic signal reflected from the subject, and converts the received ultrasonic signal into an electrical signal. In this regard, the echo signal may include the ultrasonic signal reflected from the subject and the converted electrical signal.

The RF frame acquisition unit 120 acquires at least two RF frames including a first RF frame and a second RF frame from the echo signal reflected from the subject.

For example, the RF frame acquisition unit 120 may acquire an N number of RF frames by reception beamforming on the echo signal reflected from the subject. In this regard, N may be 1 or a natural number greater than 1, and may be determined according to a speed (frame/sec) and a reception time of a reception frame. For example, if RF frames are received for 10 seconds at a speed of 30 frames per second, N may be 300.

The respective RF frames acquired by the RF frame acquisition unit 120 according to an embodiment includes the information regarding the cross-sections of the subject formed in the first direction and the second direction. That is, the respective RF frames acquired by the RF frame acquisition unit 120 may include information regarding cross-sectional images acquired at a predetermined time interval with respect to the same subject.

The displacement estimation unit 130 estimates a second directional displacement indicating a degree of movement of a first point of the subject appearing in the first RF frame in the second direction in the second RF frame.

In this regard, the first RF frame and the second RF frame may be temporally neighboring two frames among the N number of RF frames but are not limited thereto. That is, the first RF frame and the second RF Frame may be a first frame and a second frame acquired by the RF frame acquisition unit 120 but are not limited thereto. The first RF frame and the second RF Frame may be the first frame and a last frame acquired by the RF frame acquisition unit 120.

Further, the first RF frame may be acquired before heat is applied to the subject, and the second RF frame may be acquired after heat is applied to the subject but are not limited thereto.

The first RF frame and the second RF frame according to an embodiment are RF frames of the same subject, whereas the subject may not appear as being the same in the first RF frame and the second RF frame.

More specifically, if a temperature of a medium through which the ultrasonic signal passes varies, a propagation speed of the ultrasonic signal also varies. In this way, if the temperature of the medium through which the ultrasonic signal passes is not constant, the ultrasonic signal is curved due to refraction of the ultrasonic signal by a change of speed of the ultrasonic signal. In this regard, the refraction of the ultrasonic signal by a change of speed of the ultrasonic signal may correspond to thermal lens effect.

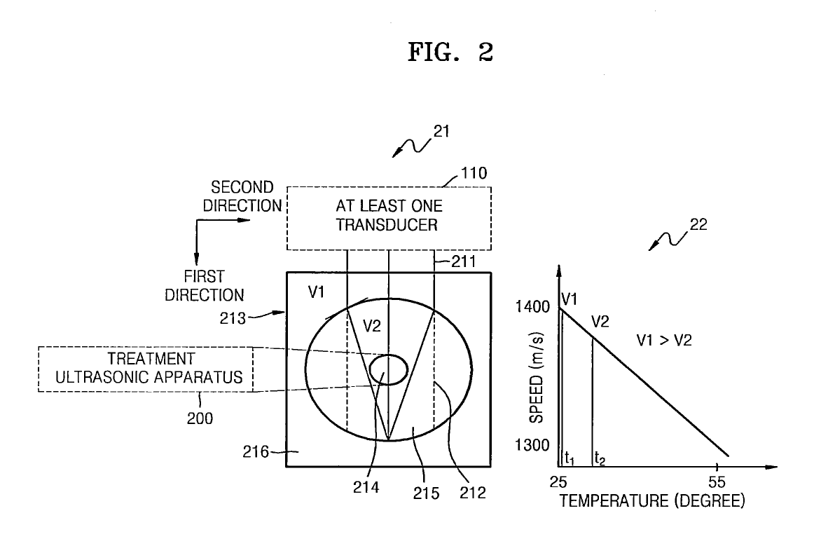

For example, when heat is applied to a treatment part of a subject, the propagation speed of the ultrasonic signal is reduced. In this regard, the treatment part may have a fat property. Accordingly, the ultrasonic signal transmitted by the at least one transducer 110 is curved in a direction neighboring the treatment part during propagating in the first direction. This will be described in detail with reference to FIG. 2 below.

In this way, since the ultrasonic signal transmitted by the at least one transducer 110 is curved, the same point of the subject may have different positions in the first RF frame and the second RF frame.

Accordingly, the displacement estimation unit 130 estimates the second directional displacement indicating the degree of movement of the first point of the subject appearing in the first RF frame in the second direction in the second RF frame.

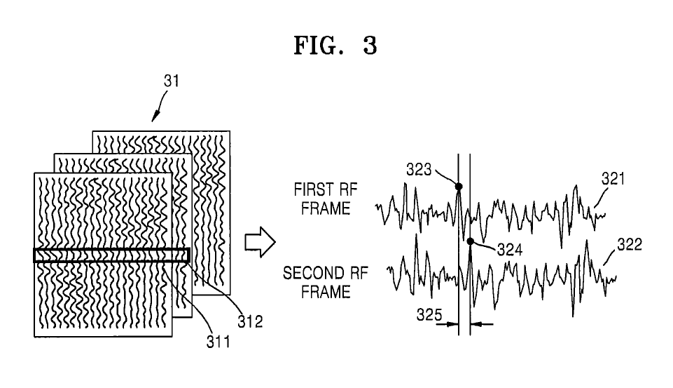

For example, the displacement estimation unit 130 compares a second direction line including the first point of the subject in the first RF frame and a second direction line including the first point of the subject in the second RF frame, and estimates the second directional displacement indicating the degree of movement of the first point of the subject appearing in the first RF frame in the second direction in the second RF frame according to a result of comparison. This will be described in detail with reference to FIG. 3 below.

For another example, the displacement estimation unit 130 may calculate cross-correlation between the first RF frame and the second RF frame, and estimate the second directional displacement indicating the degree of movement of the first point of the subject appearing in the first RF frame in the second direction in the second RF frame using the calculated cross-correlation. The cross-correlation between the first RF frame and the second RF frame according to an embodiment may be normalized cross-correlation (NCC) but is not limited thereto.

More specifically, the displacement estimation unit 130 calculates auto-correlation of the first RF frame, calculates auto-correlation of the second RF frame, and calculates the cross-correlation between the first RF frame and the second RF frame. Thereafter, the displacement estimation unit 130 may calculate the NCC of the first RF frame and the second RF frame using the calculated auto-correlation of the first RF frame, the calculated auto-correlation of the second RF frame, and the calculated cross-correlation between the first RF frame and the second RF frame.

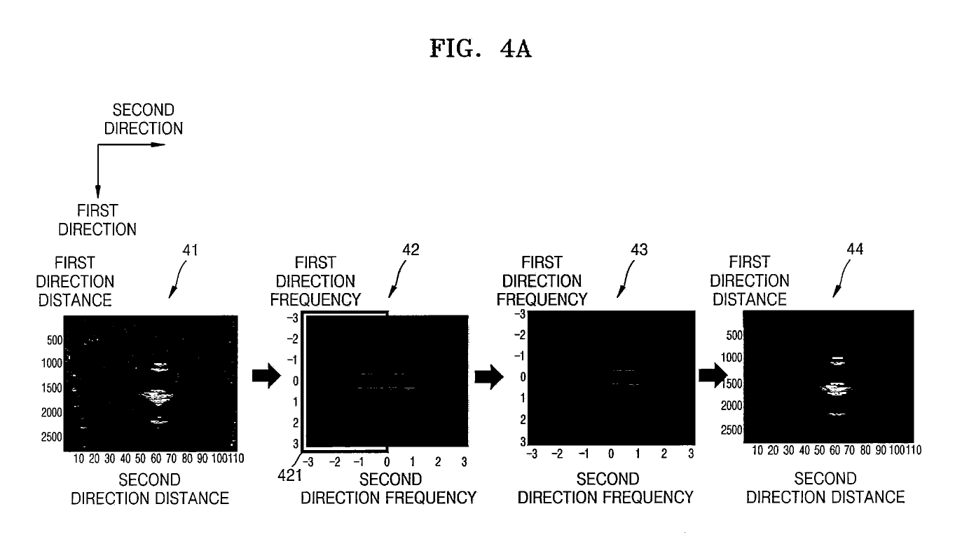

In addition, the displacement estimation unit 130 according to an embodiment may calculate the cross-correlation between the first RF frame and the second RF frame using a second direction analytic signal from which a negative frequency component with respect to the second direction is removed in order to enhance accuracy. This will be described in detail with reference to FIGS. 4A and 4B.

The displacement estimation unit 130 may estimate the second directional displacement indicating the degree of movement of the first point of the subject in the second direction in the second RF frame using the cross-correlation between the first RF frame and the second RF frame by using a speckle tracking method.

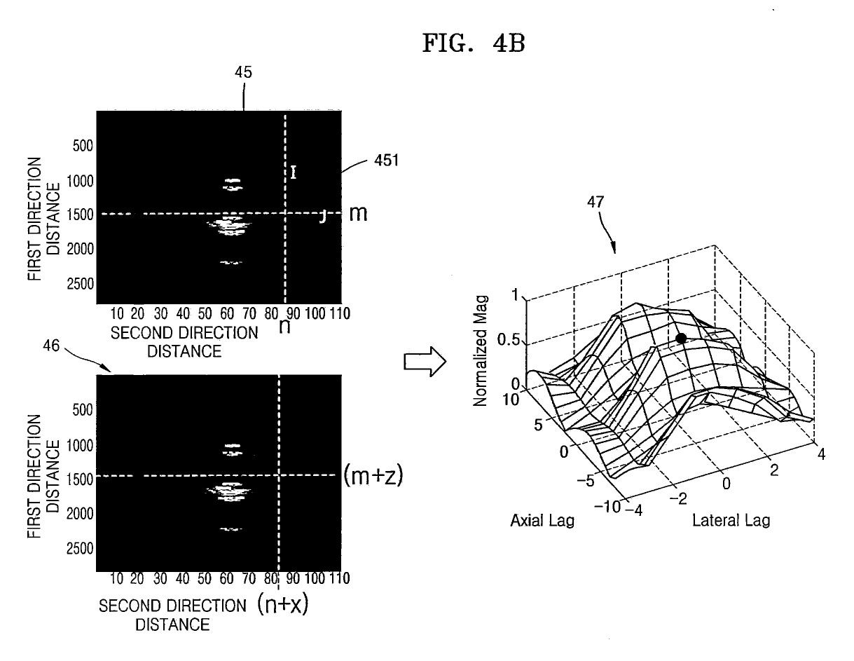

More specifically, the displacement estimation unit 130 may determine a predetermined region including the first point of the subject in the first RF frame, detect a point having the greatest cross-correlation between the first RF frame and the second RF frame from the determined region, and estimate the second directional displacement using a position of the detected point having the greatest cross-correlation in the second direction. This will be described in detail with reference to FIG. 5 below.

Alternatively, the displacement estimation unit 130 may determine a predetermined region including the first point of the subject in the first RF frame, detect a point having the greatest cross-correlation between the first RF frame and the second RF frame from the determined region, detect a point at which a phase of cross-correlation is zero-crossing with respect to the second direction line including the detected point having the greatest cross-correlation, and estimate the second directional displacement by calculating a degree of delay of the detected zero-crossing point in the second direction. This will be described in detail with reference to FIG. 5 below.

Further, the displacement estimation unit 130 may further perform parabolic interpolation before detecting the point in which cross-section phases including the detected point having the greatest cross-correlation with respect to the second direction line are zero-crossed.

Accordingly, the displacement estimation unit 130 may estimate a displacement degree of the same point of the subject between the first RF frame and the second RF frame in the second direction as the second directional displacement.

The image generation unit 140 generates an ultrasonic image corresponding to the second RF frame. Further, the image generation unit 140 may generate ultrasonic images corresponding to a plurality of frames acquired by the RF frame acquisition unit 120. In this regard, the ultrasonic image according to an embodiment may include a B-mode image, a temperature image, etc. but is not limited thereto.

For example, the image generation unit 140 may include a digital signal processor (DSP), a digital scan converter (DSC), etc. used to generate an ultrasonic image of the subject using the RF frames acquired by the RF frame acquisition unit 120 but is not limited thereto.

Further, the image generation unit 140 according to an embodiment may use a sound of speed (SOS) technique, a change in backscattered energy (CBE) technique, a B.A technique, etc. in order to generate temperature images corresponding to the RF frames acquired by the RF frame acquisition unit 120 but is not limited thereto.

The error correction unit 150 corrects an error of the ultrasonic image corresponding to the second RF frame generated by the image generation unit 140 using the second directional displacement estimated by the displacement estimation unit 130. The correcting of the error of the ultrasonic image in the error correction unit 150 according to an embodiment may include interpolating of values constituting the ultrasonic image.

For example, if the image generation unit 140 generates the temperature image corresponding to the second RF frame, the error correction unit 150 may correct an error of the temperature image using a temperature of the first point in the temperature image and a temperature of a point neighboring the first point in the second direction in the temperature image.

For example, if the image generation unit 140 generates the B-mode image corresponding to the second FR frame, the error correction unit 150 may correct an error of the B-mode image using brightness of the first point in the B-mode image and brightness of a point neighboring the first point in the second direction in the B-mode image.

Hereinafter, the ultrasonic image generated by the image generation unit 140 is the temperature image but is not limited thereto. For convenience of description, the first point of the subject is (n, m), a temperature of the first point of the subject in the temperature image corresponding to the second RF frame is T(n, m), and the second directional displacement with respect to the first point estimated by the displacement estimation unit 130 is δx(n,m).

The error correction unit 150 may correct the error of the temperature image by performing an arithmetic operation like equation 1 below if the second directional displacement δx(n,m) with respect to the first point is greater than or equal to 0 (zero), and performing an arithmetic operation like equation 2 below if the second directional displacement δx(n,m) with respect to the first point is smaller than 0 (zero).

a. [Equation 1]

δx(n,m)≧0,

T(n,m)=T(n,m)·δx(n,m)+T(n+1,m)(1−δx(n,m))

b. [Equation 2]

δx(n,m)<0,

T′(n,m)=T(n−1,m)·(1+δx(n,m))+T(n+1,m)(−δx(n,m))

Referring to equations 1 and 2 above, T′(n, m) is a temperature having a corrected error of the first point, T(n, m) is the temperature of the first point, δx(n, m) is the second directional displacement with respect to the first point, n is a position of the first point in the second direction, and m is a position of the first point in the first direction.

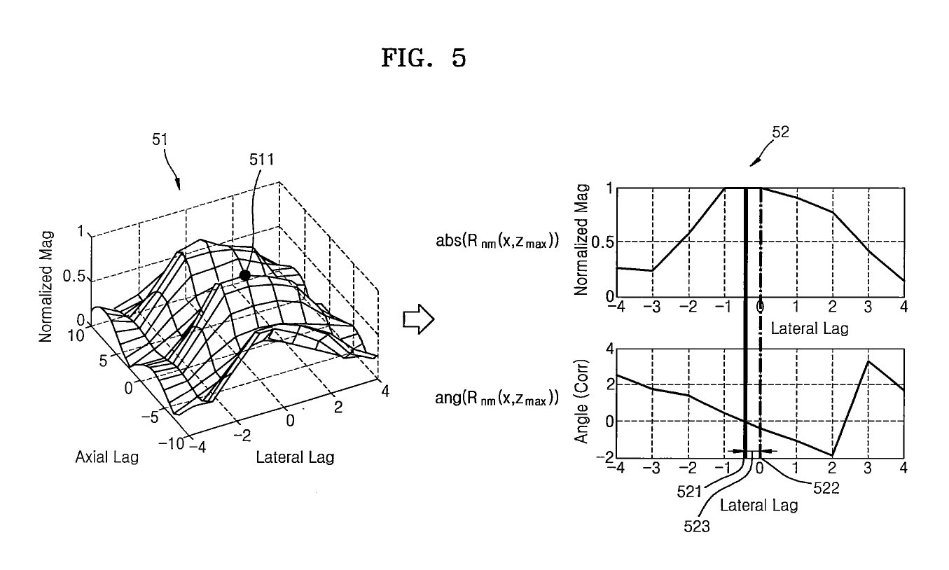

A point neighboring the first point in equations 1 and 2 is (n−1, m) or (n+1, m) but is not limited thereto. The point neighboring the first point may include all points away from the first point by a predetermined distance. In this regard, the predetermined distance may be determined by a user. The method of correcting the error in the error correction unit 150 will be described in more detail with reference to FIG. 6 below.

As such, the error correction unit 150 may correct the error of the ultrasonic image and also generate the error corrected ultrasonic image according to a result of correction.

Accordingly, the apparatus 100 for generating the diagnostic image according to an embodiment may accurately generate the diagnostic image if a temperature of the subject is not constant. More specifically, as the treatment part of the subject is heated, the subject may be prevented from looking expansive in the B-mode image and a temperature of the subject may be prevented from looking inaccurate in the temperature image.

Further, the method of correcting an error of one pixel with respect to one line of the ultrasonic image indicating the first point of the subject is explained with reference to FIG. 1 but is not limited thereto. Errors of all pixels constituting the ultrasonic image or some pixels thereof may be corrected according to settings. As such, if errors of at least two pixels constituting the ultrasonic image are corrected, the error corrected ultrasonic image may be generated by repeatedly performing the error correction method described above on a plurality of pixels or simultaneously performing the error correction method on a plurality of pixels.

In this case, the user may designate an error correction range like all pixels of the ultrasonic image or some pixels thereof. If all pixels of the ultrasonic image are corrected, an amount of arithmetic operations increases, which reduces a speed of generating the ultrasonic image, and thus the apparatus 100 for generating the diagnostic image according to an embodiment may appropriately adjust the speed of generating the ultrasonic image with respect to the increase in the amount of arithmetic operations based on user's settings.

FIG. 2 illustrates a propagation path 21 of an ultrasonic signal and a propagation speed 22 thereof with respect to a temperature of a medium, according to an embodiment. For example, the medium according to an embodiment has a fat property, and exhibits a characteristic that the higher the temperature of the medium, the slower the speed of the ultrasonic signal passing through the medium.

In the propagation path 21 of the ultrasonic signal, the at least one transducer 110 of FIG. 1 irradiates an ultrasonic transmission signal in a first direction to a subject 213.

In this regard, the subject 213 may include a treatment part 214 to which the treatment ultrasonic apparatus 200 may apply heat. Accordingly, the subject 213 may be classified into a part 215 neighboring the treatment part 214 having about t₁° C. and a part 216 not neighboring the treatment part 214 having about t₂° C. In this regard, t₁ and t₂ satisfy a condition of t₁>t₂.

The ultrasonic transmission signal transmitted by the at least one transducer 110 propagates in the first direction, and thus a display path 212 of the ultrasonic image in a dotted line is a straight line irrespective of the temperature of the subject 213.

However, as shown in the propagation speed 22 of the ultrasonic signal with respect to the temperature of the medium, since a propagation speed of an ultrasonic signal in a medium having the temperature of t₁° C. is faster than a propagation speed of the ultrasonic signal in a medium having the temperature of t₂° C., the propagation path 211 of the ultrasonic signal in a broken line is curved in a direction of the treatment part 214.

That is, if the at least one transducer 110 irradiates the ultrasonic transmission signal in the first direction to the subject 213, the propagation path 211 of the ultrasonic signal is curved in a second direction perpendicular to the first direction.

As described above, since the propagation path 211 of the ultrasonic image and the display path 212 thereof differ from each other, the ultrasonic image may be distorted unlike the actual characteristic of the subject 213. More specifically, the ultrasonic image may be distorted in the second direction perpendicular to the propagation direction of the ultrasonic signal.

Therefore, the apparatus 100 for generating the diagnostic image according to an embodiment may estimates a second directional displacement indicating a degree of movement of a point of a subject in a second direction, and generates an ultrasonic image in which an error in the second direction is corrected using the second directional displacement.

FIG. 3 is a diagram for explaining a method of estimating a displacement in the displacement estimation unit 130 of FIG. 1.

FIG. 3 shows an N number of RF frames 31 acquired by the RF frame acquisition unit 120 of FIG. 1. The displacement estimation unit 130 selects a first RF frame and a second RF frame from among the N number of RF frames 31. In this regard, the displacement estimation unit 130 may determine an ath frame and an (a+1)th frame as the first RF frame and the second RF frame among the N number of RF frames 31 but is not limited thereto.

Since a temperature of a subject is not constant, a first point of the subject has different positions in the first RF frame and the second RF frame. Accordingly, the displacement estimation unit 130 may compare a second direction line 311 including the first point of the subject in the first RF frame and a second direction line 312 including the first point of the subject in the second RF frame, and estimate a second directional displacement according to a result of comparison.

More specifically, the displacement estimation unit 130 detects the second direction lines 311 and 312 including the first point of the subject from the first RF frame and the second RF frame, respectively. The second direction lines 311 and 312 respectively detected from the first RF frame and the second RF frame may be a first signal 321 and a second signal 322.

As shown in FIG. 3, a first point 323 of the subject in the first signal 321 and a first point 324 of the subject in the second signal 322 may do not have the same position in the first RF frame and the second RF frame.

Accordingly, the displacement estimation unit 130 may compare the first signal 321 and the second signal 322, and estimate a second directional displacement 325 indicating a degree of movement of the first point 324 of the subject in the second signal 322 in the second direction according to a result of comparison.

FIG. 4A is a diagram for explaining a method of extracting a second directional analytic signal so as to calculate cross-correlation in the displacement estimation unit 130 of FIG. 1. Referring to FIG. 4A, an RF frame 41 may be one of a plurality of RF frames acquired by the RF frame acquisition unit 120. For example, the RF frame 41 may be a first RF frame or a second RF frame.

The displacement estimation unit 130 may extract the second directional analytic signal from which a negative frequency component 421 with respect to a second direction is removed so as to accurately estimate a second directional displacement with respect to a first point of a subject.

For example, the displacement estimation unit 130 converts the RF frame 41 of a time domain into an RF frame 42 of a frequency domain. In this regard, the displacement estimation unit 130 may convert the RF frame 41 of the time domain into the RF frame 42 of the frequency domain using a Fourier transform (FT) technique, a fast Fourier transform (FFT) technique, a 2D FFT technique, etc., but is not limited thereto.

Further, the displacement estimation unit 130 removes the negative frequency component 421 with respect to the second direction from the RF frame 42 of the frequency domain. In FIG. 4A, an RF frame 43 may be a frame obtained from which the negative frequency component 421 with respect to the second direction of the RF frame 42 is removed.

Further, the displacement estimation unit 130 converts the RF frame 43 of the frequency domain from which the negative frequency component 421 with respect to the second direction is removed into an RF frame 44 of the time domain. In this regard, the displacement estimation unit 130 may convert the RF frame 43 of the frequency domain into the RF frame 44 of the time domain using an inverse Fourier transform (IFT) technique, an inverse fast Fourier transform (IFFT) technique, a 2D IFFT technique, etc., but is not limited thereto.

Accordingly, the displacement estimation unit 130 may extract the second directional analytic signal from the RF frame 44. For example, if a first direction is an axis z, and a second direction is an axis x, the second directional analytic signal may be expressed as equation 3 below.

a. [Equation 3]

s(x,z)=A(x,z)e^{-jφ(x,z)}

In equation 3, s(x, z) denotes a point of the RF frame 44, A(x, z) denotes amplitude of the point s(x, z), and φ(x, z) denotes a phase of the point s(x, z).

As described above, the displacement estimation unit 130 estimates the second directional displacement using the second direction analytic signal extracted from the RF frame 44 from which the negative frequency component 421 with respect to the second direction is removed, thereby enhancing accuracy of the estimation.

FIG. 4B is a diagram for explaining a method of calculating cross-correlation between a first RF frame 45 and a second RF frame 46 in the displacement estimation unit 130 of FIG. 1.

The displacement estimation unit 130 determines a predetermined region 451 including a first point of a subject in the first RF frame 45. In this regard, the predetermined region 451 may be a 2D kernel but is not limited thereto.

For convenience of description, the predetermined region 451 is determined as an I×J size in FIG. 4B but is not limited thereto. In this regard, I and J may be 0 or a real number greater than 0.

Assumed that the first point of the subject is positioned at (n, m) and (n+x, m+z) in the first RF frame 45 and the second RF frame 46, respectively, the cross-correlation between the first RF frame 45 and the second RF frame 46 may be defined as equation 4 below.

$$\begin{matrix}[ Equation 4 ] & \\ {R}_{nm} ( x , z ) = {\sum }_{i = - I / 2}^{I / 2} {\sum }_{j = - J / 2}^{J / 2} {s}_{1} ( n + i , m + j ) {s}_{2}^{*} ( n + x + i , m + z + j ) . & a\end{matrix}$$

In equation 4, Rₙₘ(x, z) denotes the cross-correlation between the first RF frame 45 and the second RF frame 46, I and J denote a size of the predetermined region 451, and s₁( ) and s₂( ) are second directional analytic signals extracted according to equation 3 with respect to the RF frame 45 and the second RF frame 46, respectively.

Accordingly, the displacement estimation unit 130 may calculate the cross-correlation between the first RF frame 45 and the second RF frame 46 through an arithmetic operation of equation 4 above. The calculated cross-correlation may be expressed as a graph 47.

FIG. 5 is a diagram for explaining a method of estimating a displacement in the displacement estimation unit 130 of FIG. 1. FIG. 5 shows a graph 51 indicating cross-correlation between a first RF frame and a second RF frame.

With reference to the graph 51, the displacement estimation unit 130 detects a point 511 having the greatest cross-correlation between the first RF frame and the second RF frame. More specifically, the displacement estimation unit 130 detects the point 511 having the greatest cross-correlation between the first RF frame and the second RF frame calculated according to equation 4.

For convenience of description, the point 511 having the greatest cross-correlation between the first RF frame and the second RF frame detected by the displacement estimation unit 130 is (xₘₐₓ, zₘₐₓ) but is not limited thereto.

For example, the displacement estimation unit 130 may estimate a second directional displacement using a second directional position of the point 511 having the greatest cross-correlation. In this case, the second directional displacement may be xₘₐₓ.

In this regard, the calculated second directional displacement may be a displacement according to a sample resolution. That is, the second directional displacement xₘₐₓ may be calculated in a pixel unit constituting an ultrasonic image, and thus the error correction unit 150 may correct an error in the pixel unit.

For another example shown in graph 52, the displacement estimation unit 130 may detect a point 521 at which a phase of cross-correlation is zero-crossing with respect to a second direction line including the point 511 having the greatest cross-correlation between the first RF frame and the second RF frame, and estimate the second directional displacement by calculating a degree of delay of the detected zero-crossing point 521 in the second direction.

In this regard, the cross-correlation with respect to the second direction line including the point 511 having the greatest cross-correlation may be Rₙₘ(x, zₘₐₓ), and the degree of delay of the detected zero-crossing point 521 in the second direction may be a degree of delay 523 from a point 522 at which x is 0 (zero). In this case, the second directional displacement may be δx.

In this regard, the calculated second directional displacement δx may be a displacement according to a subsample resolution. For example, δx may satisfy a condition of equation 5 below.

a. [Equation 5]

−1≦δx≦1

That is, the second directional displacement δx may be calculated in a unit smaller

than the pixel unit constituting the ultrasonic image, and thus the error correction unit 150 may correct an error smaller than the pixel unit.

Accordingly, the displacement estimation unit 130 may estimate the second directional displacement indicating a degree of movement of a first point of the subject appearing in the first RF frame in the second direction in the second RF frame.

FIG. 6 is a diagram for explaining a method of correcting an error of an ultrasonic image in the error correction unit 150 of FIG. 1. For convenience of description, a temperature image is described but is not limited thereto. The method may also apply to a B-mode image.

Referring to FIG. 6, a temperature image 61 corresponds to a second RF frame generated by the image generation unit 140. For example, a first point of a subject is (98, 80), and a second point thereof is (99, 80). Further, for convenience of description, description regarding the first point and the second point of the subject is described but is not limited thereto.

The error correction unit 150 corrects errors of the first point and the second point of the subject by performing interpolation as shown in a graph 62. In this regard, the error correction unit 150 may correct errors through the arithmetic operation of equation 1 or 2 in consideration of the second directional displacement δx(n,m) estimated by the displacement estimation unit 130.

The graph 62 shows a first temperature curve 621 and a second temperature curve 622.

In this regard, the first temperature curve 621 represents temperatures of pixels constituting a second directional line 611 of the temperature image 61, and the second temperature curve 612 represents error corrected temperatures of the pixels constituting the second directional line 611. In the temperature image 61, a first directional distance between the pixels constituting the second directional line 611 may be 80 mm.

The first temperature curve 621 includes a first point 623 and a second point 624 of the subject. The second temperature curve 622 includes a first point 625 and a second point 626 of the subject.

As described above, the error correction unit 150 may apply the arithmetic operation of equation 1 or 2 to all second directional lines constituting the temperature image 61, and thus an error of the temperature image 61 may be completely corrected.

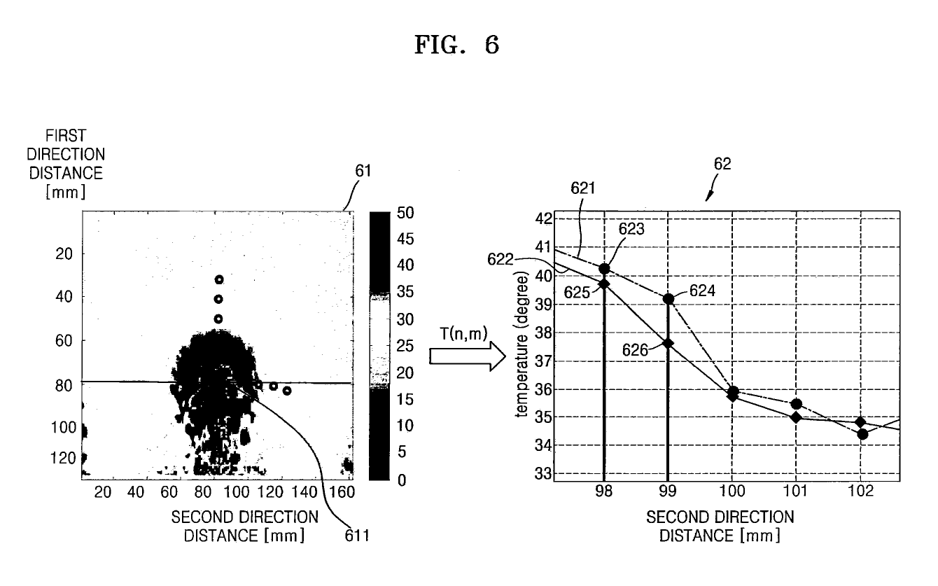

FIG. 7 is a block diagram of a medical image system 700, according to an embodiment. Referring to FIG. 7, the medical image system 700 includes the apparatus 100 for generating a diagnostic image, a storage unit 710, a display unit 720, and an output unit 730.

The apparatus 100 for generating the diagnostic image of FIG. 7 corresponds to the apparatus 100 for generating the diagnostic image of FIG. 1. Accordingly, the description with reference to FIG. 1 may be applied to the apparatus 100 for generating the diagnostic image of FIG. 1, and thus redundant descriptions will not be repeated herein.

The medical image system 700 shown in FIG. 7 includes elements related to an embodiment. A medical image system 700 may further include other general-purpose elements.

The diagnostic image generating apparatus 100 for generating the diagnostic image transmits a transmission signal to a subject in a first direction, acquires a first RF frame and a second RF frame from an echo signal reflected from the subject, estimates a second directional displacement indicating a degree of movement of a first point of the subject appearing in the first RF frame in a second direction perpendicular to the first direction in the second RF frame, and generates an error corrected ultrasonic image using the estimated second directional displacement.

The storage unit 710 stores the diagnostic image generated by the apparatus 100 for generating the diagnostic image. The display unit 720 displays the diagnostic image generated by the apparatus 100 for generating the diagnostic image. However, the medical image system 700 according to this embodiment does not include the display unit 720 but includes the output unit 730 for outputting the diagnostic image generated by the apparatus 100 for generating the diagnostic image to an external display apparatus (not shown).

The output unit 730 outputs the diagnostic image generated by the apparatus 100 for generating the diagnostic image to an external apparatus over a wired or wireless network or through wired serial communication. For example, the external apparatus may include a universal serial bus (USB) memory, a general-purpose computer system, a remotely located medical image system, a facsimile machine, a portable terminal, a personal digital assistant (PDA), etc.

The output unit 730 may transmit and receive data to and from the external apparatus over a wired or wireless network. A network according to an embodiment may include the Internet, a local area network (LAN), a wireless LAN, a wide area network (WAN), or a personal area network (PAN), but is not limited thereto as long as it transmits and receives information.

Accordingly, an image reading and searching function may be further included in the storage unit 720 and the output unit 730 so as to integrate the storage unit 710 and the output unit 730 as a picture archiving communication system (PACS).

Accordingly, the medical image system 700 may store, display, and output the error corrected ultrasonic image generated by the apparatus 100 for generating the diagnostic image to the external apparatus.

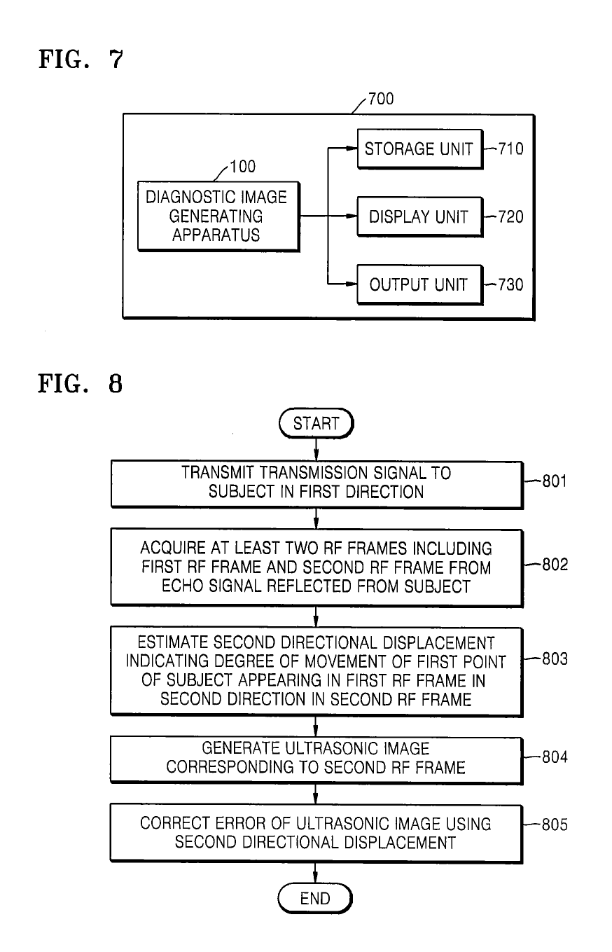

FIG. 8 is a flowchart illustrating a method of generating a diagnostic image, according to an embodiment. Referring to FIG. 8, the method includes operations performed in time-series by the apparatus 100 for generating the diagnostic image of FIG. 1 or the medical image system 700 of FIG. 7. Thus, the description regarding the apparatus 100 for generating the diagnostic image of FIG. 1 or the medical image system 700 of FIG. 7 may also be applied to the method of FIG. 8, even if it is omitted.

In operation 801, the at least one transducer 110 transmits a transmission signal to a subject in a first direction. In this regard, the subject may include a treatment part to which heat is applied.

In operation 802, the RF frame acquisition unit 120 acquires at least two RF frames including a first RF frame and a second RF frame from an echo signal reflected from the subject.

In operation 803, the displacement estimation unit 130 estimates a second directional displacement indicating a degree of movement of a first point of the subject appearing in the first RF frame in a second direction in the second RF frame. In this regard, the second direction may be perpendicular to the first direction that is a propagation direction of the transmission signal.

In operation 804, the image generation unit 140 generates an ultrasonic image corresponding to the second RF frame. In this regard, the ultrasonic image may be planar to the first direction and the second direction.

In operation 805, the error correction unit 150 corrects an error of the ultrasonic image generated in operation 804 using the second directional displacement estimated in operation 803. Accordingly, the error correction unit 150 may generate the error corrected ultrasonic image.

According to an embodiment, if a temperature of the subject is not constant, an accurate ultrasonic image may be generated.

If the temperature of the subject is not constant, the inaccurate ultrasonic image may prevent from being generated due to a variable speed of the ultrasonic signal passing through the subject.

Furthermore, if a tissue is dead using a HIFU system, the apparatus 100 for generating the diagnostic image according to an embodiment may generate an accurate temperature image even when the temperature of the subject is not constant, thereby accurately performing temperature monitoring. Accordingly, reliability of the HIFU system may be enhanced.

As described above, according to the one or more of the above embodiments an ultrasonic image may be accurately generated by using an echo signal reflected from a subject.

Processes, functions, methods, and/or software in apparatuses described herein may be recorded, stored, or fixed in one or more non-transitory computer-readable storage media (computer readable recording medium) that includes program instructions (computer readable instructions) to be implemented by a computer to cause one or more processors to execute or perform the program instructions. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of the computer readable recording medium include a magnetic storage media (e.g., ROM, RAM, USB, floppy disks, hard disks, etc.), optical recording media (e.g., CD-ROMs, or DVDs), and a PC interface (e.g., PCI, PCI-express, WIFI, etc.) Examples of program instructions include both machine code, such as produced by a compiler, and files including higher level code that may be executed by the computer using an interpreter. The described hardware devices may be configured to act as one or more software modules that are recorded, stored, or fixed in one or more computer-readable storage media, in order to perform the operations and methods described above, or vice versa. In addition, a non-transitory computer-readable storage medium may be distributed among computer systems connected through a network and computer-readable codes or program instructions may be stored and executed in a decentralized manner. In addition, the computer-readable storage media may also be embodied in at least one application specific integrated circuit (ASIC) or Field Programmable Gate Array (FPGA)

Although embodiments have been shown and described, it would be appreciated by those skilled in the art that changes may be made in these embodiments without departing from the principles and spirit of the disclosure, the scope of which is defined in the claims and their equivalents.

What is claimed is:

1. A method of generating a diagnostic image with respect to a first direction and a second direction of a subject, the method comprising:
transmitting a transmission signal in the first direction to the subject;
acquiring at least two radio frequency (RF) frames including a first RF frame and a second RF frame from an echo signal reflected from the subject;
estimating a second directional displacement indicating a degree of movement of a first point of the subject appearing in the first RF frame in a second direction in the second RF frame;
generating an ultrasonic image corresponding to the second RF frame; and
correcting an error of the generated ultrasonic image using the estimated second directional displacement.

2. The method of claim 1, wherein the subject comprises a treatment part to which heat is applied.

3. The method of claim 1, wherein:
the generating of the ultrasonic image comprises generating a temperature image corresponding to the second RF frame; and
the correcting of the error comprises correcting an error of the temperature image using a temperature of the first point in the temperature image and a temperature of a point neighboring the first point in the second direction in the temperature image.

4. The method of claim 1, wherein:
the generating of the ultrasonic image comprises generating a brightness (B)-mode image corresponding to the second RF frame; and
the correcting of the error comprises correcting an error of the B-mode image using brightness of a first point in the B-mode image and brightness of a point neighboring the first point in the second direction in the B-mode image.

5. The method of claim 1, wherein the estimating of the second directional displacement comprises comparing a second directional line including the first point in the first RF frame and a second directional line including the first point in the second RF frame and estimating the second directional displacement according to a result of comparison.

6. The method of claim 1, wherein the estimating of the second directional displacement comprises calculating cross-correlation between the first RF frame and the second RF frame and estimating the second directional displacement using the calculated cross-correlation.

7. The method of claim 6, wherein the estimating of the second directional displacement comprises estimating the directional second displacement using the calculated cross-correlation by using a second directional analytic signal from which a negative frequency component with respect to the second direction is removed.

8. The method of claim 6, wherein the estimating of the second directional displacement comprises:
determining a predetermined region including the first point of the subject in the first RF frame;
detecting a point having the greatest cross-correlation between the first RF frame and the second RF frame from the determined region; and
estimating the second directional displacement using a position of the detected point having the greatest cross-correlation in the second direction.

9. The method of claim 6, wherein the estimating of the second directional displacement comprises:
determining a predetermined region including the first point of the subject in the first RF frame, detecting a point having the greatest cross-correlation between the first RF frame and the second RF frame from the determined region;
detecting a point at which a phase of cross-correlation is zero-crossing with respect to a second direction line including the detected point having the greatest cross-correlation; and
estimating the second directional displacement by calculating a degree of delay of the detected zero-crossing point in the second direction.

10. A non-transitory computer readable recording medium having recorded thereon computer readable instructions that control at least one processor to implement the method of claim 1.

11. An apparatus for generating an ultrasonic a diagnostic image with respect to a first direction and a second direction of a subject, the apparatus comprising:
at least one transducer to transmit a transmission signal in the first direction to the subject and receiving an echo signal reflected from the subject;
a radio frequency (RF) frame acquisition unit to acquire at least two RF frames including a first RF frame and a second RF frame from the echo signal;
a displacement estimation unit to estimate a second directional displacement indicating a degree of movement of a first point of the subject appearing in the first RF frame in a second direction in the second RF frame;
an image generation unit to generate an ultrasonic image corresponding to the second RF frame; and
an error correction unit to correct an error of the generated ultrasonic image using the estimated second directional displacement.

12. The apparatus of claim 11, further comprising: a treatment ultrasonic apparatus to apply heat to a treatment part of the subject.

13. The apparatus of claim 11, wherein the image generation unit generates a temperature image corresponding to the second RF frame, and the error correction unit corrects an error of the temperature image using a temperature of the first point in the temperature image and a temperature of a point neighboring the first point in the second direction in the temperature image.

14. The apparatus of claim 11, wherein the displacement estimation unit calculates cross-correlation between the first RF frame and the second RF frame and estimates the second directional displacement using the calculated cross-correlation.

15. The apparatus of claim 14, wherein the displacement estimation unit determines a predetermined region including the first point of the subject in the first RF frame, detects a point having the greatest cross-correlation between the first RF frame and the second RF frame from the determined region, and estimates the second directional displacement using a position of the detected point having the greatest cross-correlation in the second direction.

16. The apparatus of claim 14, wherein the displacement estimation unit determines a predetermined region including the first point of the subject in the first RF frame, detects a point having the greatest cross-correlation between the first RF frame and the second RF frame from the determined region, detects a point at which a phase of cross-correlation is zero-crossing with respect to a second direction line including the detected point having the greatest cross-correlation, and estimates the second directional displacement by calculating a degree of delay of the detected zero-crossing point in the second direction.

17. A medical image system comprising:
an apparatus to generate a diagnostic image by transmitting a transmission signal in a first direction to a subject, to acquire a first radio frequency (RF) frame and a second RF frame from an echo signal reflected from the subject, to estimate a directional displacement indicating a degree of movement of a first point of the subject appearing in the first RF frame in a second direction perpendicular to the first direction in the second RF frame, and to generate an error corrected ultrasonic image using the estimated directional displacement; and
a display unit to display the generated error corrected ultrasonic image.

18. The medical image system of claim 17, further comprising: a treatment ultrasonic apparatus to apply heat to a treatment part of the subject.

19. The medical image system of claim 17, wherein the apparatus to generate the diagnostic image generates a temperature image corresponding to the second RF frame, and generates the error corrected ultrasonic image using a temperature of the first point in the temperature image and a temperature of a point neighboring the first point in the second direction in the temperature image.

20. The medical image system of claim 17, wherein the apparatus to transmit the diagnostic image determines a predetermined region including the first point of the subject in the first RF frame, to detect a point having the greatest cross-correlation between the first RF frame and the second RF frame from the determined region, to detect a point at which a phase of cross-correlation is zero-crossing with respect to a second direction line including the detected point having the greatest cross-correlation, and to estimate the second directional displacement by calculating a degree of delay of the detected zero-crossing point in the second direction.
